(19) United States
(12) Patent Application Publication
(10) Pub. No.: US 2025/0367532 A1
(43) Pub. Date: Dec. 4, 2025

(54) APPROACHES TO PROVIDING PERSONALIZED FEEDBACK ON PHYSICAL ACTIVITIES BASED ON REAL-TIME ESTIMATION OF POSE AND SYSTEMS FOR IMPLEMENTING THE SAME

(71) Applicant: Hinge Health, Inc., San Francisco, CA (US)

(72) Inventors: Colin Joseph Brown, Saskatoon (CA); Alexander Peplowski, Montreal (CA); Sacha Terzian, Montreal (CA); Louis Harbour, Montreal (CA); Maxime Gill-Comeau, Montreal (CA)

(21) Appl. No.: 19/299,618

(22) Filed: Aug. 14, 2025

Related U.S. Application Data
(63) Continuation of application No. PCT/US24/16513, filed on Feb. 20, 2024.
(60) Provisional application No. 63/486,226, filed on Feb. 21, 2023.

(51) Int. Cl. A63B 71/06; A63B 24/00; G06V 10/75; G06V 10/764; G06V 40/10; G06V 40/20

(52) U.S. Cl. CPC A63B 71/0622; A63B 24/0062; G06V 10/751; G06V 10/764; G06V 40/10; G06V 40/23; A63B 2024/0068; A63B 2220/05; A63B 2230/62

(57) ABSTRACT

Introduced here are computer-implemented platforms (also referred to as “motion monitoring platforms”) that are able to provide feedback in a personalized manner during the performance of physical activities. By monitoring the current state of an individual while performing a physical activity, a motion monitoring platform can more readily identify feedback that is likely to have its intended effect.

### CROSS-REFERENCE TO RELATED APPLICATIONS

This application is a continuation of International Application No. PCT/US2024/016513, filed Feb. 20, 2024, entitled “APPROACHES TO PROVIDING PERSONALIZED FEEDBACK ON PHYSICAL ACTIVITIES BASED ON REAL-TIME ESTIMATION OF POSE AND SYSTEMS FOR IMPLEMENTING THE SAME” which claims priority to U.S. Provisional Application No. 63/486,226, entitled “Approaches to Providing Personalized Feedback on Physical Activities based on Real-Time Estimation of Pose and Systems for Implementing the Same” and filed on Feb. 21, 2023, each of which is incorporated by reference herein in its entirety.

### TECHNICAL FIELD

Various embodiments concern computer programs and associated computer-implemented techniques for estimating pose of a living body and providing appropriate feedback to promote completion of physical activities.

### BACKGROUND

Pose estimation (also called “pose detection”) is an active area of study in the field of computer vision. Over the last several years, tens—if not hundreds—of different approaches have been proposed in an effort to solve the problem of pose detection. Many of these approaches rely on machine learning due to its programmatic approach to learning what constitutes a pose.

As a field of artificial intelligence, computer vision enables machines to perform image processing tasks with the aim of imitating human vision. Pose estimation is an example of a computer vision task that generally includes detecting, associating, and tracking the movements of a person. This is commonly done by identifying “key points” that are semantically important to understanding pose. Examples of key points include “head,” “left shoulder,” “right shoulder,” “left knee,” and “right knee.” Insights into posture and movement can be drawn from analysis of these key points.

### BRIEF DESCRIPTION OF THE DRAWINGS

FIG. 1 includes several examples of generalized feedback.

FIG. 2 illustrates a network environment that includes a motion monitoring platform that is executed by a computing device.

FIG. 3 illustrates an example of a computing device that is able to execute a motion monitoring platform.

FIG. 4 includes a high-level diagrammatic illustration of a process for recognizing different stages of a physical activity (here, an exercise).

FIG. 5 includes a high-level diagrammatic illustration of a process for providing feedback during the different states of the physical activity.

FIG. 6 includes illustrations of different states for several examples of physical activities (here, a clamshell stretch and squat).

FIG. 7 includes an exemplary schema of a six-state machine that can be used to recognize the different states of a physical activity.

FIG. 8 includes a flow of red-green-blue (“RGB”) digital images that illustrate how the motion monitoring platform can estimate the raw pose and then forward align the raw poses.

FIG. 9 illustrates how a template can be captured by estimating the pose in a video where an expert (e.g., a physiotherapist) showcases the ideal movement and, in some embodiments, undesired variations for which feedback is to be provided.

FIG. 10 includes an example of estimated poses being matched against the template prepared for a given physical activity (here, a squat).

FIG. 11 includes a block diagram illustrating an example of a processing system in which at least some operations described herein can be implemented.

Various features of the technology described herein will become more apparent to those skilled in the art from a study of the Detailed Description in conjunction with the drawings. Various embodiments are depicted in the drawings for the purpose of illustration. However, those skilled in the art will recognize that alternative embodiments may be employed without departing from the principles of the technology. Accordingly, although specific embodiments are shown in the drawings, the technology is amenable to various modifications.

### DETAILED DESCRIPTION

Over the last several years, significant advances have been made in the field of computer vision. This has resulted in the development of sophisticated pose estimation programs (also called “pose estimators” or “pose predictors”) that are designed to perform pose estimation in either two dimensions or three dimensions. Two-dimensional (“2D”) pose estimators predict the 2D spatial locations of key points, generally through the analysis of the pixels of a single digital image. Three-dimensional (“3D”) pose estimators predict the 3D spatial arrangement of key points, generally through the analysis of the pixels of multiple digital images, for example, consecutive frames in a video, or a single digital image in combination with another type of data generated by, for example, an inertial measurement unit (“IMU”) or Light Detection and Ranging (“LiDAR”) unit.

Pose estimators-both 2D and 3D-continue to be applied to different contexts, and as such, continue to be used to help solve different problems. One problem for which pose estimators have proven to be particularly useful is monitoring the performance of physical activities. Consider, for example, a scenario where an individual is instructed or prompted to perform a physical activity by a computer program. By applying a pose estimator to digital images of the individual, the computer program can glean insight into the performance of the physical activity. Historically, the individual may have instead been asked to summarize her performance of the physical activity (e.g., in terms of difficulty); however, this type of manual feedback tends to be inaccurate and inconsistent. Due to their consistent, programmatic nature, pose estimators allow for more accurate monitoring of performances of physical activities.

This is especially important if the pose estimator is responsible for monitoring physical activities that have meaningful real-world impact, such as on the health and wellness of the individual responsible for performing the physical activities. Exercise therapy is an intervention technique that utilizes physical activities as the principal treatment for addressing the symptoms of musculoskeletal (“MSK”) conditions, such as acute physical ailments and chronic physical ailments. Exercise therapy programs (or simply “programs”) generally involve a plan for performing physical activities during exercise therapy sessions (or simply “sessions”) that occur on a periodic basis. Normally, the purpose of a program is to either restore normal MSK functionality or reduce the pain caused by a physical ailment, which may have been caused by injury or disease.

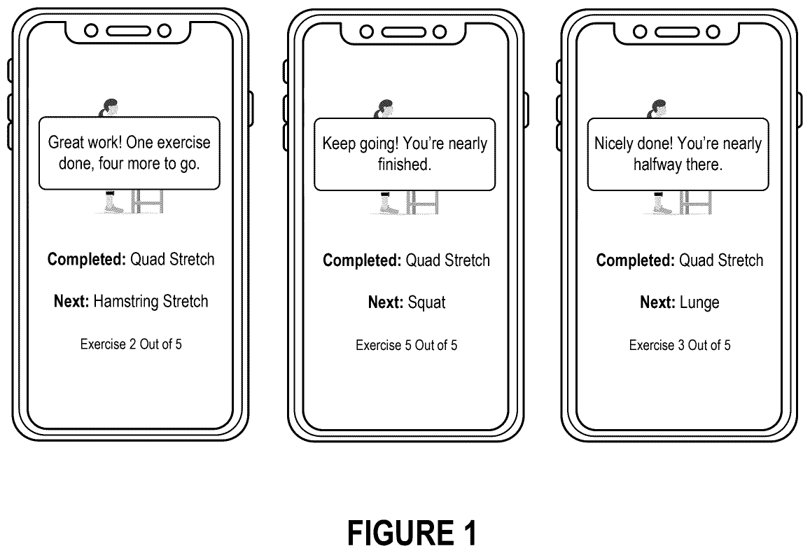

Programs generally explain, either audibly or visually, how an individual (also called a “user,” “patient,” or “participant”) should perform physical activities to achieve a therapeutic goal. However, individuals can—and often do—struggle to adhere to their respective programs unless consistently engaged. One approach to engagement involves contacting individuals outside of sessions, for example, via text messages that indicate when a next session is to be completed. Another approach to engagement involves offering feedback during sessions. While there is some benefit to offering generalized feedback—examples of which are shown in FIG. 1—many individuals either do not respond to generalized feedback or quickly become “immune” to generalized feedback.

Introduced here is an approach to providing feedback in a personalized manner during the performance of physical activities. The approach not only can help solve the problem of accurately counting repetitions of physical activities but can also provide useful feedback without requiring that a healthcare professional (e.g., physiotherapist, nurse, or physician) be present when the repetitions are being performed. Simply put, the approach allows individuals to perform high-quality exercise therapy at home.

As further discussed below, the approach may rely on real-time analysis of poses that are estimated for an individual as she performs a physical activity. These estimated poses—or indicia that are visually representative thereof—may be presented for display on an interface that is accessible via a computing device. Generally, the computing device is associated with the individual and is responsible for generating the digital images from which the poses are estimated.

Given a series of representations of the estimated pose of the individual over time, a motion monitoring platform can:
Extract one or more salient features of the individual;
Drive a cyclical state machine that represents repetitions of the physical activity;
Initiate transitions in state, which may be used to count repetitions, based on generic rules such as matching extracted poses to an established template for the physical activity;
Match other state-specific conditions, if any, that may queue relevant feedback; and
Prioritize, trigger, and present queued feedback in a way that is helpful for the individual.

The nature of the representations may depend on the nature of the pose extractor that is applied by the motion monitoring platform to produce the series of representations. For example, if the pose extractor is a 2D pose extractor, the representations may be 2D skeletal frames that define the 2D spatial locations of key points. If the pose extractor is a 3D pose extractor, the representations may be 3D skeletal frames that define the 3D spatial locations of key points.

One benefit is that this approach may be generic to a large variety of physical activities, though specific parameters and templates can be defined per physical activities. Accordingly, a set of algorithms corresponding to different physical activities could be developed and then released for the motion monitoring platform, but additional algorithms corresponding to new physical activities could be added to the set or existing algorithms corresponding to existing physical activities could be removed from the set.

For the purpose of illustration, embodiments may be described with reference to exercises that are performed during sessions as part of a program. However, the motion monitoring platform could be designed to monitor performance of other physical activities, such as sporting activities, cooking activities, art activities, and the like. Accordingly, the approach described herein could be used to provide personalized feedback regarding performance of nearly any physical activity.

Moreover, embodiments may be described in the context of computer-executable instructions for the purpose of illustration. However, aspects of the approach could be implemented via hardware or firmware instead of, or in addition to, software. As an example, the motion monitoring platform may be embodied as a computer program that offers support for completing exercises during sessions as part of a program, determines which physical activities are appropriate for a user given performance during past sessions, and enables communication between the user and one or more coaches. The term “coach” may be used to generally refer to individuals who prompt, encourage, or otherwise facilitate engagement by users with the motion monitoring platform. Coaches are generally not healthcare professionals but could be in some embodiments.

### Terminology

References in the present disclosure to “an embodiment” or “some embodiments” mean that the feature, function, structure, or characteristic being described is included in at least one embodiment. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense. That is, in the sense of “including but not limited to.” The term “based on” is also to be construed in an inclusive sense. Thus, the term “based on” is intended to mean “based at least in part on.”

The terms “connected,” “coupled,” and variants thereof are intended to include any connection or coupling between two or more elements, either direct or indirect. The connection or coupling can be physical, logical, or a combination thereof. For example, elements may be electrically or communicatively coupled to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing all tasks.

When used in reference to a list of multiple items, the word “or” is intended to cover all of the following interpretations: any of the items in the list, all of the items in the list, and any combination of items in the list.

### Overview of Motion Monitoring Platform

A motion monitoring platform may be responsible for monitoring the motion of an individual (also called a “user,” “patient,” or “participant”) through analysis of digital images that contain her and are captured as she completes a physical activity. As an example, the motion monitoring platform may guide the user through exercise therapy sessions (or simply “sessions”) that are performed as part of an exercise therapy program (or simply “program”) by monitoring pose in an ongoing manner. As part of the program, the user may be requested to engage with the motion monitoring platform on a periodic basis. The frequency with which the user is requested to engage with the motion monitoring platform may be based on factors such as the anatomical region for which therapy is needed, the MSK condition for which therapy is needed, the difficulty of the program, the age of the user, the amount of progress that has been achieved, and the like. Note that because the motion of the user is generally monitored through the continual analysis of pose, the motion monitoring platform could also be called a “pose monitoring platform.”

As the user performs exercises, she may be recorded by a camera of a computing device. Normally, the camera is part of the computing device on which the motion monitoring is executed or accessed. For example, in order to initiate a session, the user may initiate a mobile application that is stored on, and executable by, her mobile phone or tablet computer, and the mobile application may instruct the user to position her mobile phone or tablet computer in such a manner that one of its cameras can record her as exercises are performed. Note that, in some embodiments, the camera is part of another computing device. For example, the camera may be included in a peripheral computing device, such as a web camera (also called a “webcam”), that is connected to the computing device. By examining the digital images that are output by the camera, the motion monitoring platform can monitor performance of the exercises by estimating the pose of the user over time.

As mentioned above, the motion monitoring platform could alternatively estimate pose in contexts that are unrelated to healthcare, for example, to improve technique. As an example, the motion monitoring platform may estimate the pose of an individual while she completes a sporting activity (e.g., performs a dance move, performs a yoga move, shoots a basketball, throws a baseball, swings a golf club), a cooking activity, an art activity, etc. Accordingly, while embodiments may be described in the context of a user who completes an exercise during a session as part of a program, the features of those embodiments may be similarly applicable to individuals performing other types of physical activities. Individuals whose performances of physical activities are analyzed may be referred to as “users” of the motion monitoring platform, even if these individuals have little to no opportunity to interact with the motion monitoring platform.

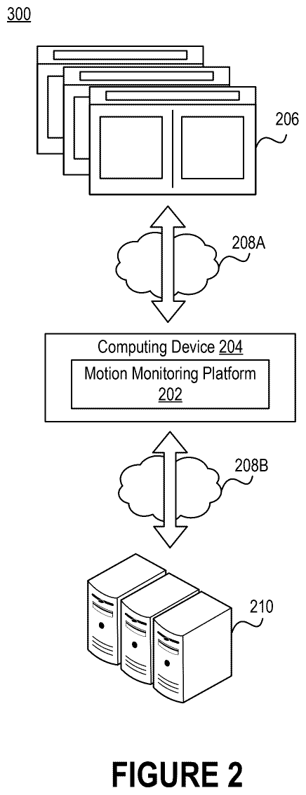

FIG. 2 illustrates a network environment 200 that includes a motion monitoring platform 202 that is executed by a computing device 204. Users can interact with the motion monitoring platform 202 via interfaces 206. For example, users may be able to access interfaces that are designed to guide them through physical activities, indicate progress, present feedback, etc. As another example, users may be able to access interfaces through which information regarding completed physical activities can be reviewed, feedback can be provided, etc. Thus, interfaces 206 may serve as informative spaces, or the interfaces 206 may serve as collaborative spaces through which users and coaches can communicate with one another.

As shown in FIG. 2, the motion monitoring platform 202 may reside in a network environment 200. Thus, the computing device on which the motion monitoring platform 202 is executing may be connected to one or more networks 206A-B. Depending on its nature, the computing device 204 could be connected to a personal area network (“PAN”), local area network (“LAN”), wide area network (“WAN”), metropolitan area network (“MAN”), or cellular network. For example, if the computing device 204 is a mobile phone, then the computing device 204 may be connected to a computer server of a server system 210 via the Internet. As another example, if the computing device 204 is a computer server, then the computing device 204 may be accessible to users via respective computing devices that are connected to the Internet via LANs.

The interfaces 206 may be accessible via a web browser, desktop application, mobile application, or another form of computer program. For example, to interact with the motion monitoring platform 202, a user may initiate a web browser on the computing device 204 and then navigate to a web address associated with the motion monitoring platform 202. As another example, a user may access, via a desktop application or mobile application, interfaces that are generated by the motion monitoring platform 202 through which she can select physical activities to complete, review analyses of her performance of the physical activities, and the like. Accordingly, interfaces generated by the motion monitoring platform 202 may be accessible via various computing devices, including mobile phones, tablet computers, desktop computers, wearable electronic devices (e.g., watches or fitness accessories), virtual reality systems, augmented reality systems, and the like.

Generally, the motion monitoring platform 202 is hosted, at least partially, on the computing device 204 that is responsible for generating the digital images to be analyzed, as further discussed below. For example, the motion monitoring platform 202 may be embodied as a mobile application executing on a mobile phone or tablet computer. In such embodiments, the instructions that, when executed, implement the motion monitoring platform 202 may reside largely or entirely on the mobile phone or tablet computer. Note, however, that the mobile application may be able to access a server system 210 on which other aspects of the motion monitoring platform 202 are hosted.

In some embodiments, aspects of the motion monitoring platform 202 are executed by a cloud computing service operated by, for example, Amazon Web Services®, Google Cloud Platform™, or Microsoft Azure®. Accordingly, the computing device 204 may be representative of a computer server that is part of a server system 210. Often, the server system 210 comprises multiple computer servers. These computer servers can include information regarding different physical activities; computer-implemented models (or simply “models”) that indicate how anatomical regions should move when a given physical activity is performed; computer-implemented templates (or simply “templates”) that indicate how anatomical regions should be positioned when partially or fully engaged in a given physical activity; algorithms for processing image data from which spatial position of anatomical regions can be computed, inferred, or otherwise determined; user data such as name, age, weight, ailment, enrolled program, duration of enrollment, and number of physical activities completed; and other assets.

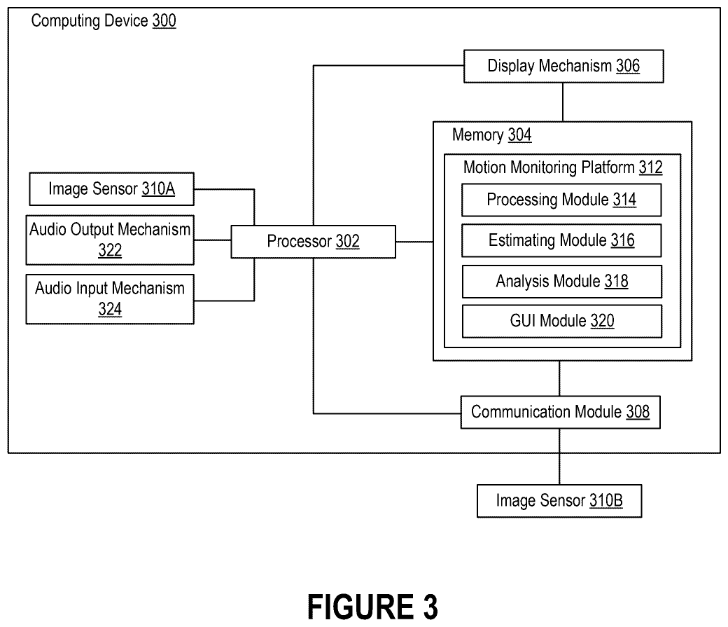

FIG. 3 illustrates an example of a computing device 300 that is able to execute a motion monitoring platform 312. As mentioned above, the motion monitoring platform 312 can facilitate the performance of physical activities by a user, for example, by providing instruction or encouragement. As shown in FIG. 3, the computing device 300 can include a processor 302, memory 304, display mechanism 308, communication module 308, image sensor 310A, audio output mechanism 322, and audio input mechanism 324. Each of these components is discussed in greater detail below.

Those skilled in the art will recognize that different combinations of these components may be present depending on the nature of the computing device 300. For example, if the computing device 300 is a computer server that is part of a server system (e.g., server system 210 of FIG. 2), then the computing device 300 may not include the display mechanism 306, image sensor 310A, audio output mechanism 322, or audio input mechanism 324, though the computing device 200 may be communicatively connectable to another computing device that does include a display mechanism, an image sensor, an audio output mechanism, or an audio input mechanism.

The processor 302 can have generic characteristics similar to general-purpose processors, or the processor 302 may be an application-specific integrated circuit (“ASIC”) that provides control functions to the computing device 300. As shown in FIG. 3, the processor 302 can be coupled to all components of the computing device 300, either directly or indirectly, for communication purposes.

The memory 304 may be comprised of any suitable type of storage medium, such as static random-access memory (“SRAM”), dynamic random-access memory (“DRAM”), electrically erasable programmable read-only memory (“EEPROM”), flash memory, or registers. In addition to storing instructions that can be executed by the processor 302, the memory 304 can also store data generated by the processor 302 (e.g., when executing the modules of the motion monitoring platform 312) and produced, retrieved, or obtained by the other components of the computing device 300. For example, data received by the communication module 308 from a source external to the computing device 300 (e.g., image sensor 310B) may be stored in the memory 304, or data produced by the image sensor 310A may be stored in the memory 304. Note that the memory 304 is merely an abstract representation of a storage environment. The memory 304 could be comprised of actual integrated circuits (also referred to as “chips”).

The display mechanism 306 can be any mechanism that is operable to visually convey information to a user. For example, the display mechanism 306 may be a panel that includes light-emitting diodes (“LEDs”), organic LEDs, liquid crystal elements, or electrophoretic elements. In some embodiments, the display mechanism 306 is touch sensitive. Thus, a user may be able to provide input to the motion monitoring platform 312 by interacting with the display mechanism 306. Alternatively, the user may be able to provide input to the motion monitoring platform 312 through some other control mechanism.

The communication module 308 may be responsible for managing communications external to the computing device 300. For example, the communication module 308 may be responsible for managing communications with other computing devices (e.g., server system 210 of FIG. 2, or a camera peripheral such as video camera or webcam). The communication module 308 may be wireless communication circuitry that is designed to establish communication channels with other computing devices. Examples of wireless communication circuitry include 2.4 gigahertz (“GHz”) and 5 GHz chipsets compatible with Institute of Electrical and Electronics Engineers (“IEEE”) 802.11—also referred to as “Wi-Fi chipsets.” Alternatively, the communication module 308 may be representative of a chipset configured for Bluetooth®, Near Field Communication (“NFC”), and the like. Some computing devices—like mobile phones and tablet computers—are able to wirelessly communicate via separate channels. Accordingly, the communication module 308 may be one of multiple communication modules implemented in the computing device 300. As an example, the communication module 308 may initiate and then maintain one communication channel with a camera peripheral (e.g., via Bluetooth), and the communication module 308 may initiate and then maintain another communication channel with a server system (e.g., via the Internet).

The nature, number, and type of communication channels established by the computing device 300—and more specifically, the communication module 308—may depend on the sources from which data is received by the motion monitoring platform 312 and the destinations to which data is transmitted by the motion monitoring platform 312. Assume, for example, that the computing device 400 is representative of a mobile phone or tablet computer that is associated with (e.g., owned by) a user. In some embodiments the communication module 308 may only externally communicate with a computer server, while in other embodiments the communication module 308 may also externally communicate with a source from which to receive image data. The source could be another computing device (e.g., a mobile phone or camera peripheral that includes an image sensor 310B) to which the mobile device is communicatively connected. Image data could be received from the source even if the mobile phone generates its own image data. Thus, image data could be acquired from multiple sources, and these image data may correspond to different perspectives of the user performing a physical activity. Regardless of the number of sources, image data—or analyses of the image data—may be transmitted to the computer server for storage in a digital profile that is associated with the user. The same may be true if the motion monitoring platform 312 only acquires image data generated by the image sensor 310A. The image data may initially be analyzed by the motion monitoring platform 312, and then the image data—or analyses of the image data—may be transmitted to the computer server for storage in the digital profile.

The image sensor 310A may be any electronic sensor that is able to detect and convey information in order to generate images, generally in the form of image data (also called “pixel data”). Examples of image sensors include charge-coupled device (“CCD”) sensors and complementary metal-oxide semiconductor (“CMOS”) sensors. The image sensor 310A may be part of a camera module (or simply “camera”) that is implemented in the computing device 300. In some embodiments, the image sensor 310A is one of multiple image sensors implemented in the computing device 300. For example, the image sensor 310A could be included in a front- or rear-facing camera on a mobile phone. Alternatively, the image sensor 310A may be externally connected to the computing device 300 such that the image sensor 310A captures image data of an environment and sends the image data to the motion monitoring platform 312.

For convenience, the motion monitoring platform 312 may be referred to as a computer program that resides in the memory 304. However, the motion monitoring platform 312 could be comprised of hardware or firmware in addition to, or instead of, software. In accordance with embodiments described herein, the motion monitoring platform 312 may include a processing module 314, pose estimating module 316, analysis module 318, and graphical user interface (“GUI”) module 320. These modules can be an integral part of the motion monitoring platform 312. Alternatively, these modules can be logically separate from the motion monitoring platform 312 but operate “alongside” it. Together, these modules may enable the motion monitoring platform 312 to programmatically monitor motion of users during the performance of physical activities, such as exercises, through analysis of digital images generated by the image sensor 310.

The processing module 314 can process image data obtained from the image sensor 310A over the course of a session. The image data may be used to infer a spatial position or orientation of one or more anatomical regions as further discussed below. The image data may be representative of a series of digital images. These digital images may be discretely captured by the image sensor 310A over time, such that each digital image captures the user at different stages of performing a physical activity. In some embodiments, these digital images may be representative of frames of a video that is captured by the image sensor 310. In such embodiments, the image data could also be called “video data.”

The image data may be used to infer a spatial position of one or more anatomical regions as further discussed below. For example, the processing module 314 may perform operations (e.g., filtering noise, changing contrast, reducing size) to ensure that the data can be handled by the other modules of the motion monitoring platform 312. As another example, the processing module 314 may temporally align the data with data obtained from another source (e.g., another image sensor) if multiple data are to be used to establish the spatial position of the anatomical regions of interest.

Moreover, the processing module 314 may be responsible for processing information input by users through interfaces generated by the GUI module 320. For example, the GUI module 320 may be configured to generate a series of interfaces that are presented in succession to a user as she completes physical activities as part of a session. On some or all of these interfaces, the user may be prompted to provide input. For example, the user may be requested to indicate (e.g., via a verbal command or tactile command provided via, for example, the display mechanism 306) that she is ready to proceed with the next physical activity, that she completed the last physical activity, that she would like to temporarily pause the session, etc. These inputs can be examined by the processing module 314 before information indicative of these inputs is forwarded to another module.

The pose estimating module 316 (or simply “estimating module”) may be responsible for estimating the pose of the user through analysis of image data, in accordance with the approach further discussed below. Specifically, the estimating module 316 can create, based on a digital image (e.g., generated by the image sensor 310A or image sensor 310B), a skeletal frame that specifies a spatial position of each of multiple anatomical regions. For example, the estimating module 316 can apply a computer-implemented model (or simply “model”) called a pose estimator to the digital image, so as to produce the skeletal frame. In some embodiments the pose estimator is designed and trained to identify a predetermined number and/or type of anatomical regions (e.g., left and right wrist, left and right elbow, left and right shoulder, left and right hip, left and right knee, left and right ankle, or any combination thereof), while in other embodiments the pose estimator is designed and trained to identify all anatomical regions of a certain type (e.g., all joints) that are visible in the digital image provided as input. The pose estimator could be a neural network that when applied to the digital image, analyzes the pixels to independently identify digital features that are representative of each anatomical region of interest.

The analysis module 318 may be responsible for establishing the locations of anatomical regions of interest based on the outputs produced by the estimating module 316. Referring again to the aforementioned examples, the analysis module 316 could establish the locations of joints based on an analysis of the skeletal frame. Moreover, the analysis module 318 may be responsible for determining appropriate feedback for the user based on the outputs produced by the estimating module 316, in accordance with the approach further discussed below. Specifically, the analysis module 318 may determine an appropriate personalized recommendation for the user based on her current position, and a determination as to how her current position compares to a template that is associated with the physical activity that she has been instructed to perform.

Other modules could also be included in some embodiments. For example, the motion monitoring platform 312 may include a training module (not shown) that is responsible for training the pose estimator that is employed by the pose estimating module 316. As another example, the motion monitoring platform 312 may include a template generating module (not shown) that is responsible for generating templates that are used by the analysis module 318 to determine which recommendations, if any, are appropriate for a user given her current position.

Similarly, other components could be implemented in, or accessible to, the computing device 300 in some embodiments. For example, some embodiments of the computing device 300 include an audio output mechanism 322 and/or an audio input mechanism 324. The audio output mechanism 322 may be any apparatus that is able to convert electrical impulses into sound. One example of an audio output mechanism is a loudspeaker (or simply “speaker”). Meanwhile, the audio input mechanism 324 may be any apparatus that is able to convert sound into electrical impulses. One example of an audio input mechanism is a microphone. Together, the audio output and input mechanisms 322, 324 may enable feedback, such as personalized recommendation as further discussed below, to be audibly provided to the user. Assume, for example, that the user has been instructed to perform a physical activity while being recorded by the image sensor 310A. In such a scenario, the user may be audibly encouraged—in a personalized manner—via the audio output mechanism 322.

### Monitoring Physical Activities and Determining Personalized Recommendations

Various attempts have been made to improve engagement in programs that require performance of physical activities on a periodic basis. Consider, for example, an exercise therapy program that requires exercises be performed by an individual to achieve a therapeutic goal. The individual may be consistently notified, for example, via text message, email message, or push notification, but the individual may still struggle to adhere to the exercise therapy program. Simply put, because this feedback is not tailored or personalized in any way, the individual may quickly become “immune” to this feedback.

Introduced here is an approach to providing feedback in a personalized manner during the performance of physical activities. With this approach, there are several advantages over conventional approaches that rely on generalized feedback.

First, the motion monitoring platform may implement a generic state machine to model physical activities, and the generic state machine may assume a limited number of states-making computations faster and less computationally intense. For example, the generic state machine may be programmed to assume only (i) a relaxed state, (ii) an engaged state, and (iii) a semi-engaged state. As discussed above, the generic state machine could be programmed for various numbers of states, and the number of states may vary for different physical activities. Transitions between states may be defined by generic sets of conditions that can be automatically composed, inferred, or otherwise derived by the motion monitoring platform. This approach enables data-driven definitions of physical activities that can be quickly defined and validated by experts (e.g., healthcare professionals, such as physiotherapists). Note that the term “generic,” in this context, may be used to refer to a state machine that is generic across different physical activities.

Second, the motion monitoring platform can utilize a template-based approach to match locations of key points against different reference poses, so as to determine which state a user is currently in—or at least is closest to. As further discussed below, these reference poses can be captured or determined as part of a template generation operation in which the pose estimator is applied to digital images that capture a reference performance of a given physical activity. If the physical activity is an exercise, for example, the reference performance may be completed by a physiotherapist. This approach to developing and applying templates enables rapid scaling, by allowing an expert to perform a physical activity at least once and then having the ideal poses for each state of the physical activity to be extracted and set as criteria for repetition counting in an automated way.

One benefit of this template-based approach is that the motion monitoring platform can account for the bias that has historically been introduced by manual programming. Traditionally, in order to determine whether a user has completed a physical activity, the locations of different anatomical regions were compared to reference locations. However, these reference locations were rarely defined by an appropriate expert (e.g., a physiotherapist if the physical activity is an exercise), and even if these reference locations were defined by an appropriate expert, each reference location is representative of a guess as to where the corresponding anatomical region should be located during a performance of the physical activity. Here, the template can be generated based on analysis of an actual performance of the physical activity that is performed by an appropriate expert, and as such, the reference poses determined for the various states are more reliably authentic.

Moreover, this template-based approach allows the motion monitoring platform to account for the bias that has historically been introduced by computer vision. As mentioned above, the motion monitoring platform may apply, to a digital image, a pose estimator that determines spatial locations of anatomical regions of a human body. Due to the nature of its programming and training, the pose estimator may have bias in how the spatial locations are determined. If the outputs of the pose estimator are compared to generic rules, this bias cannot be accounted for. However, if the outputs of the pose estimator are compared to a template that is also based on outputs of the pose estimator, this bias can be accounted for, at least in the sense that the template may also be influenced by this bias.

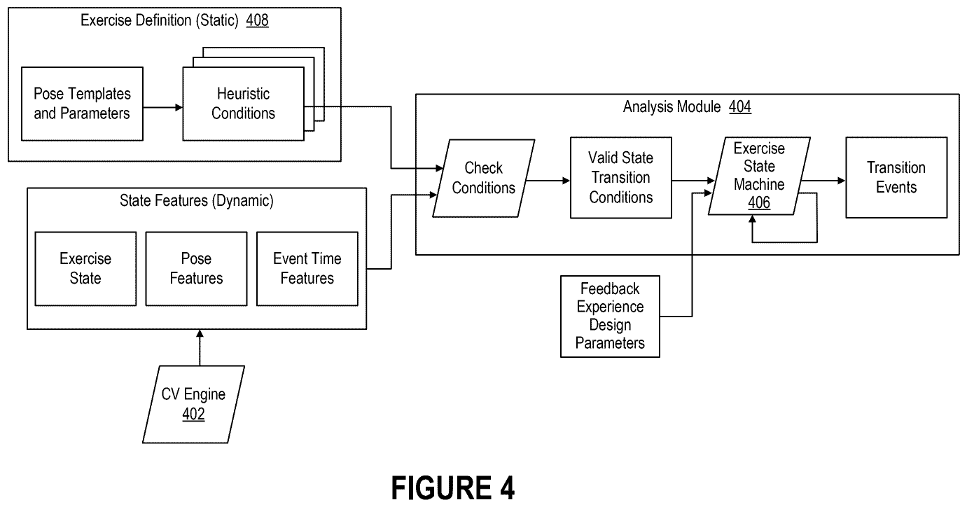

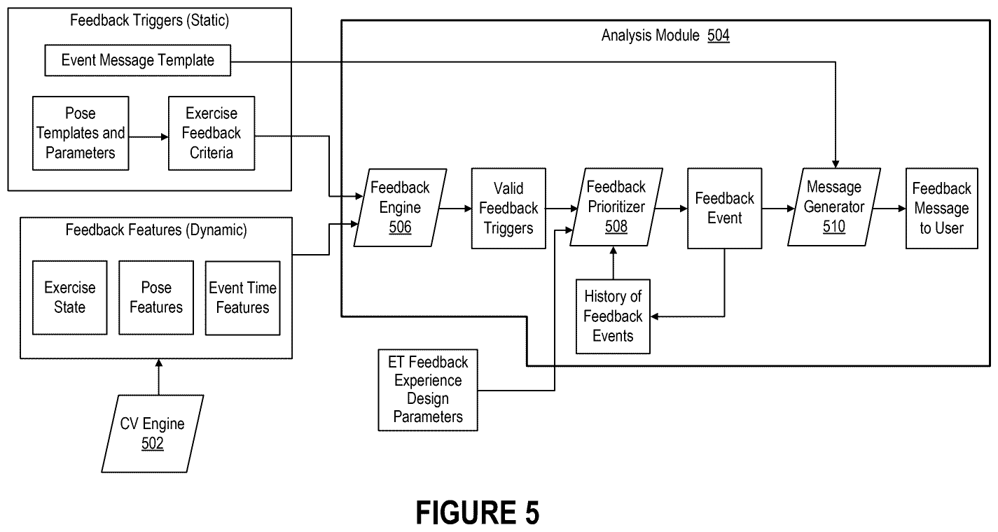

FIG. 4 includes a high-level diagrammatic illustration of a process for recognizing different stages of a physical activity (here, an exercise). FIG. 5 includes a high-level diagrammatic illustration of a process for providing feedback during the different states of the physical activity. These processes involve the motion monitoring platform using a computer vision engine (also called a “CV engine”) 402, 502 to estimate poses of a human body that is viewable in digital images, feature extraction algorithms (also called “feature extractors”) to expose relevant features of the poses corresponding to different states, and an analysis module 404, 504 (e.g., the analysis module 318 of FIG. 3) to check the definition of the stored (i.e., static) template for a physical activity and then initiate feedback based on an analysis of the features of the current state of the human body, stored parameters related to the overall user experience for the physical activity, and events that are propagated to the user.

The CV engine 402, 502 and feature extractors may be employed by the analysis module 404, 504. Alternatively, the CV engine 402, 502 may be employed by a pose estimating module (e.g., the estimating module 316 of FIG. 3). The CV engine 402, 502 may be implemented in software, firmware, hardware, or a combination thereof. Examples of CV engines include OpenPose, MediaPipe, Kinect, and proprietary CV engines developed for estimating pose. When applied to a digital image, the CV engine 402, 502 may extract relevant features of human bodies included therein, such as the 2D or 3D positions of key points, 2D shape information, 3D shape information, surface information, and the like.

Set forth below is a summary of the approach for employing a template-based approach to monitoring the location of anatomical regions of interest and determining appropriate personalized feedback. At a high level, the approach may involve the identification of physical activities (FIG. 4), such as determining repetitions or holds of short exercises, and the identification of appropriate personalized feedback (FIG. 5), such as form feedback or encouragement feedback.

As mentioned above, the processes shown in FIGS. 4-5 may involve the use of a CV engine 402, 502 that is applied to digital images to estimate the poses of a user in those digital images. Moreover, the analysis module may use (i) a feature extractor to expose relevant features of the current state of the user, (ii) a feedback engine 506 (also called a “feedback checker” or “condition checker”) to check the definition of the feedback criteria (also called “feedback triggers”) against the current state of the user, (iii) stored parameters of the overall user experience for the physical activity, and (iv) events that can be propagated to the user.

A physical activity state machine 406 (also called an “exercise state machine” or simply “state machine”) may be defined as a system, implemented in software, firmware, or hardware, that can be in one of a set number of stable conditions-referred to as “states”-depending on its previous state and the present value(s) of its input(s). Specifically, the state machine 406 may contain conditions that model the relevant states of a human body performing a given physical activity. The number of states associated with the given physical activity may depend on the difficulty of the given physical activity and total range of motion required, among other things. For example, for a simple repetitive physical activity-like an exercise that requires holding a pose-a set of states may include (i) an engaged state that corresponds to the user having achieved a valid pose for the exercise, (ii) a pre-engagement state that corresponds to the user moving into the valid pose, and (iii) a post-engagement state that corresponds to the user moving out of the valid pose. While the number of states included in a set generally is no less than three, a set could include more than three states.

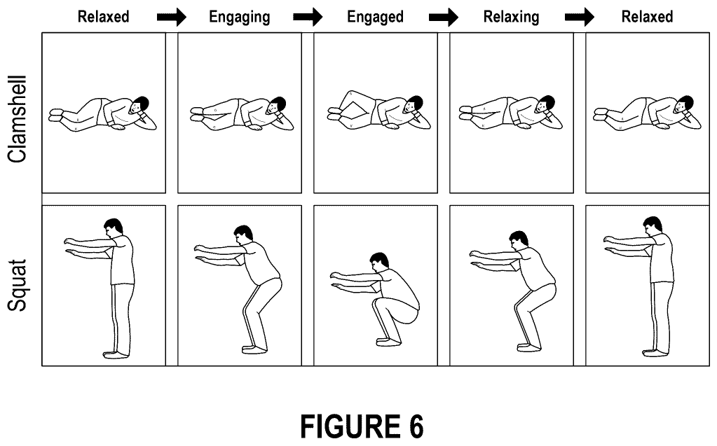

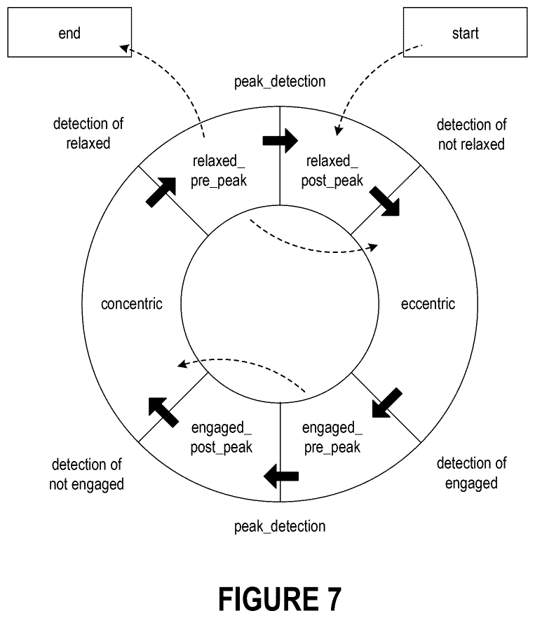

A set could include between four and twelve states, each of which corresponds to a different temporal position and/or a different spatial position with reference to a corresponding physical activity. The actual number of states in a set may depend on, for example, the speed with which the state machine 406 is expected to run or the amount of computational resources available to the state machine 406. Generally, a higher number of states is preferred because greater insight into the performance of the physical activity can be gleaned, though a higher number of states will also increase the computational resources needed by the state machine 406. Accordingly, sets may preferably include between four and eight states to balance these competing interests. However, a set of states could include up to twelve states as mentioned above. For example, a set of states may include (i) an engaged state that corresponds to the user having achieved a valid engaged pose for a physical activity, (ii) an engaged pre-extremum state that corresponds to the user having achieved the valid engaged pose but not having achieved her personal maximal engagement with the valid engaged pose, (iii) an engaged post-extremum state that corresponds to the user having achieved her personal maximal engagement and beginning to return to a valid relaxed pose, (iv) a relaxed state that corresponds to the user beginning to disengage from the valid engaged pose but not yet in what would be considered the valid relaxed pose, (v) a relaxed pre-extremum pose that corresponds to the user having achieved the valid relaxed pose but not having achieved her personal maximum engagement with the valid relaxed pose, (vi) a relaxed post-extremum pose that corresponds to the user having achieved her personal maximal engagement and holding the valid relaxed pose, (vii) a start state, and (viii) an end state. Broadly, the term “engaging” may be used to refer to a user moving towards the valid engaged pose for the physical activity but not yet having achieved the valid engaged pose (e.g., bending knees and hips in an attempt to perform a squat). Meanwhile, the term “relaxing” may be used to refer to a user beginning to disengage from a valid engaged pose and return to a valid relaxed pose. Accordingly, whereas the engaged pre-extremum and post-extremum states may be used to describe scenarios where a user is bending her knees and hips to perform a squat, the related pre-extremum and post-extremum states may be used to describe scenarios where the user is nearly fully standing and fully standing after having performed the squat. The distinction of the pre-extremum and post-extremum states may be made to facilitate execution or presentation of transition events to the user that signal whether her maximal poses have been reached while maintaining the ability to recognize repetitions with a permissive set of conditions on the engaged and relaxed states that can generalize to a large population of users with different abilities and movement styles. FIG. 6 includes illustrations of different states for several examples of physical activities (here, a clamshell stretch and squat). Meanwhile, FIG. 7 includes an exemplary schema of a six-state machine that can be used to recognize the different states of a physical activity. This schema is provided solely for the purpose of illustration, as embodiments of the state machine employed by the motion monitoring platform could include fewer than six states or more than six states. By following the exemplary schema in a clockwise manner, beginning with the start graphic, one can see how the state machine can sequentially transition between a predetermined number of states, in a predetermined order, to establish how well a physical activity is performed.

The term “transition event” may be used to refer to any event that can be exposed, by the analysis module 404, 504, to the rest of the motion monitoring platform or directly to the user to indicate a transition in the state of a physical activity. For example, transition events may be used to identify when the requirements for engagement have been met, identify the maximal engagement of a physical activity, identify when to increment a repetition counter upon relaxing following engagement, record an event notifying the completion of all repetitions for a given physical activity, and the like. Transition events may be strictly internal events to either notify other modules of the motion monitoring platform of state or be associated with audible or visual cues, such as a sound effect that serves as a notification of the completion of a set of repetitions or a visual effect that serves as a visualization of movement through the engaged state.

A data structure that is called a “physical activity definition,” “exercise definition,” or simply “definition” 408 may be stored in the memory and contain information about how the physical activity is defined. If the physical activity is an exercise, for example, the definition may include metadata about the type of exercise, preferred user state and placement within the view of the camera, and a list of heuristic conditions that define the conditions for specific state transitions within that exercise. A heuristic condition may contain some description of one or more state features (e.g., a specific pose encoded in a template, a specific joint position or flexion angle, a current state, a previous state, a time in seconds within a given state, a number of complete repetitions of the exercise, a list of other heuristic conditions), a mathematical condition on those state features (e.g., the value of the feature or some metric determined from the feature being less than, equal to, or greater than a threshold, a comparison between the values of two state features), or a score that may be based on the degree of acceptance of the aforementioned mathematical condition and may be used to rank valid conditions.

Pose features, mentioned above, may be specific poses that are encoded as 2D or 3D coordinates, for example, in a standardized coordinate system with the human body facing towards the +Z direction, with +Y representing the up direction (i.e., such that key points associated with the head will have larger Y-values than key points associated with the feet for standing poses), and +X facing the right direction. For a 3D coordinate system, the coordinates may be relative to an origin defined at the center of the pelvis of the human body (or another part of the human body, the camera, or some arbitrary global origin in space), with measurements scaled by a standardized human template (e.g., at 180 centimeters). In this case, poses of uses may be compared to established template poses for physical activities in a standardized coordinate system. Pose features may instead be encoded as joint flexion angles (in degrees or radians) of combinations of key points—also called “key joints”—or joint rotations (in quaternions), for example, relative to the pelvis or some global orientation. Pose features may instead be encoded as bone directions in 2D or 3D space, sets of distances between joints, or as principal components values in an established principal component analysis (“PCA”) based vector space (or vectors in some other feature embedding space that may be statistically or geometrically derived). These kinds of pose features may require the use of training data over which to define a population distribution of valid forms per physical activity and may help the system better generalize to not-yet-observed users. Pose features may also represent velocities, accelerations, or other time-dependent quantities that quantify the trajectory or movement of a user's poses over time.

Pose features may be used individually, for example, as single values (e.g., coordinate values, joint angle values, etc.), relative to threshold(s), or pose features may be used in combination, for example, using logical operators (e.g., AND, OR, AND/AND) or as a group to a template pose, which itself may be represented by some post features (e.g., joint coordinates, angles, velocities, etc.), and compared with an appropriate metric to compare two templates. Templates may represent a single pose of a group of poses, such as a statistical group of poses or a region of poses in an appropriate feature space.

### A Template Matching Scoring

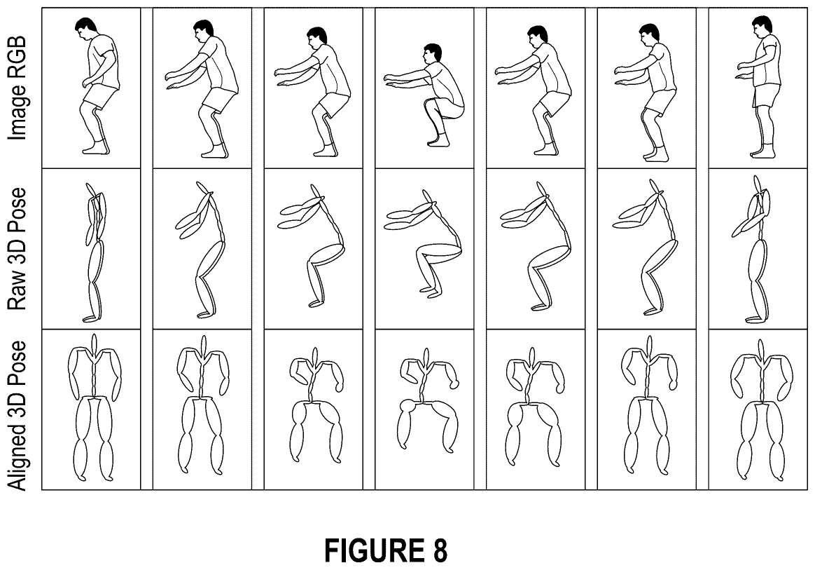

For each digital image (e.g., each frame of a video), the motion monitoring platform (and more specifically, its analysis module) can employ a matching algorithm that goes through the following steps:
Step One: Pose estimator estimates the 2D or 3D pose of a user in the digital image.
Step Two: Estimated pose is aligned such that the hips are flat in the camera plan. FIG. 8 includes a flow of red-green-blue (“RGB”) digital images that illustrate how the motion monitoring platform can estimate the raw pose and then forward align the raw poses.
Step Three: For the aligned pose, the selected pose feature(s) are computed based on the corresponding definition and features absent from a “key_features” list are masked.

### Example

$$Squat Pose Features = 3 D Joint Location$$

$$Excluded\_Features = Elblow\_L , Elbow\_R , Wrist\_L , Wrist\_R$$

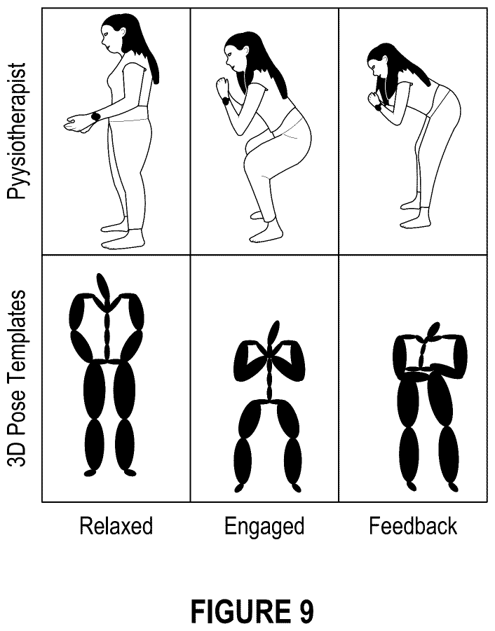

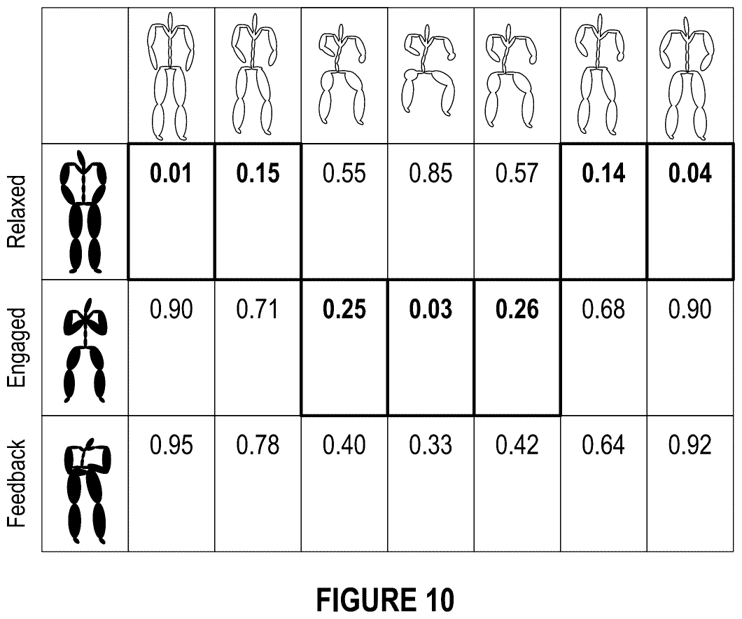

Step Four: Given a metric function, a statistical distance (or simply “distance”) is computed between the aligned pose and one or more corresponding templates that are created or captured at exercise creation time. The distance is representative of a similarity measure, namely, a real-valued function that is able to quantify similarity (e.g., between the current pose of a user and a pose corresponding to a given state of a physical activity). Depending on how those poses are represented in terms of data structure, different metric functions can be used. For poses stored as 3D coordinates with a common frame of reference (e.g. from the hips), an example of a pose score could be the Euclidean distance between the coordinates of the two poses, which goes to zero only when the two poses match. Another example could be the Euclidean distance between corresponding joint flexion angles measured on the two poses. FIG. 9 illustrates how a template can be captured by estimating the pose in a video where an expert (e.g., a physiotherapist) showcases the ideal movement and, in some embodiments, undesired variations for which feedback is to be provided. FIG. 10 includes an example of estimated poses being matched against the template prepared for a given physical activity (here, a squat). For each digital image, the state with the lowest distance can be selected as shown in FIG. 10.
Step Five: The estimated pose is classified to the state with the lowest template distance.

For the purpose of illustration, assume that the motion monitoring platform is interested in establishing statistical similarity between a pose estimated for a user and each of multiple reference poses included in a template for a physical activity. For each of the multiple reference poses, the motion monitoring platform can produce an output (also called a “distance,” “score,” or “metric”) that indicates statistical similarity to the estimated pose. Examples of these scores can be seen in FIG. 10. As mentioned above, each of the multiple reference poses may be representative of anatomical regions that are “stitched” together to form a skeletal frame. Similarly, the estimated pose may be representative of anatomical regions that are “stitched” together to form a skeletal frame. Accordingly, each score may be based on the degree of similarity of a given anatomical region (e.g., a joint) across the skeletal frames being compared. As an example, a score produced for a first reference pose may be based on a sum (e.g., a weighted sum) of sub-scores, each of which indicates similarity between a different one of multiple anatomical regions across the first reference pose and the estimated pose.

### B. Transition Events Based On Template Scores

A state machine may be designed and trained to support a large number of different exercises. Consider, for example, the six-state machine shown in FIG. 7. Such a state machine will require six positive transition events to complete a full cycle, and therefore a complete repetition of a physical activity. Transition events may be classified as either static or kinematic. Static transition events include detection of engaged and detection of relaxed, while kinematic transition events include detection of not related, peak detection in engaged, detection of not engaged, and peak detection in relaxed.

Static transition events generally depend only on data and distance scores of the current digital image. Thus, static transition events may depend on rules that cannot rely on other digital images. Examples of such rules for Detection of Engaged and Detection of Relaxed:
Minimum value of a given pose feature (e.g., knee flexion<130 for squat_engaged);
Comparing pose feature values (e.g., Ankle_L height>Ankle_R height for single_leg_balance_left);
Pose template comparison (e.g., distance (current_pose, squat_engaged_template)<70); and
Engagement value (e.g., where engagement is the difference between the related and engaged score, engaged if engagement>0 and relaxed if engagement if <0).

Kinematic transition events generally depend on the rate of change of data and distance scores, and therefore may utilize at least two consecutive digital images to make a decision. Examples of such rules for Detection of Not Relaxed and Detection of Not Engaged:
Pose velocity is large (e.g., pose velocity is computed by comparing the current pose with the previous pose, or over a smoothing window, such that the pose velocity>threshold);
Relaxed or engaged score velocity is large (e.g., the template score velocity can be computed by comparing the current template scores with previous template scores, such that relaxed velocity or engaged velocity>threshold); and
Engagement velocity is large (e.g., the engagement velocity can be computed by comparing the current engagement with the previous engagement such that engagement velocity>threshold).

Examples of such rules for Peak Detection in Engaged and Peak Detection in Relaxed:
Pose velocity at peak (e.g., pose velocity is computed by comparing the current pose with the previous pose, or over a smoothing window, such that the pose velocity is roughly zero);
Relaxed or engaged score velocity at peak (e.g., the template score velocity can be computed by comparing the current template scores with previous template scores, such that relaxed velocity or engaged velocity is roughly zero); and
Engagement velocity at peak (e.g., the engagement velocity can be computed by comparing the current engagement with the previous engagement such that engagement velocity is roughly zero).

### C. Feedback Engine

The feedback engine 506 that is implemented by the motion monitoring platform (and more specifically, its analysis module) may store and check feedback triggers, for example, one heuristic condition criterion at a time, by examining features of the user's current pose, current state, or other features. Heuristic condition criteria for a feedback trigger may include a threshold of deviation of the user's pose from an established pose template for that state, a threshold of matching between the user's pose to a feedback-specific pose template or other learned or defined rules that may be composed to identify an opportunity to provide feedback. This may generate a set of feedback triggers that are valid for the current frame. Based on the desired experience (e.g., stored in parameters) and which feedback events have already been displayed to the user (e.g., as cataloged in a history of feedback events), a separate algorithm called the “feedback prioritizer” 508 may decide which feedback events to trigger for the current digital image, if any. The history of feedback events may only be persisted per session, and therefore may be stored locally in memory and then erased from memory following the conclusion of each session.

Finally, when a feedback event is created, a feedback message may be generated by another algorithm called the “message generator” 510 and then presented to the user. The motion monitoring platform may generate the feedback message based on an event message template (or simply “message template”) that is stored in memory and any relevant dynamic state (e.g., angle of a specific joint relative to the template) in order to create a message that is relevant and personalized to the user. Note that not all feedback events may be dynamic in this way or even based on human pose. Some feedback events may simply be triggered, for example, on a timer to motivate the user. The feedback prioritizer 508 may prioritize certain types of feedback triggers over others.

### D. Summary

Accordingly, the motion monitoring platform may not only define templates for physical exercises but can also use those templates to monitor progression as users are asked to perform those physical exercises.

To define a template for a physical exercise, the motion monitoring platform can initially obtain a video that is representative of a series of frames, in temporal order, in which an individual—who may be a physiotherapist, for example—performs a physical activity. The video may be recorded by the individual in response to a determination that a template does not yet exist for the physical activity. To record the video, the individual may indicate through an interface generated by the motion monitoring platform that she is interested in defining the template. Thereafter, the motion monitoring platform can apply, to the video, a pose estimator so as to produce a series of estimated poses, each of which is representative of a pose of the individual in a corresponding one of the series of frames. The motion monitoring platform can then derive a template for the physical activity based on the series of frames. The template may include a plurality of reference poses, each of which corresponds to a different one of the estimated poses and is representative of a different state. For example, the template may include (i) a first reference pose, selected from among the estimated poses, that corresponds to a relaxed state and (ii) a second reference pose, selected from among the estimated poses, that corresponds to an engaged state. Accordingly, not all of the estimated poses—and therefore, not all of the frames—may be used to define the template. The motion monitoring platform can then store the template in a data structure or perform some other action (e.g., transmit the template to computer programs executing on computing devices associated with individuals that may be prompted to perform the physical activity).

Further, the motion monitoring platform may associate metadata with the data structure, and that metadata may specify a characteristic of the physical activity (e.g., a type of the physical activity, an intensity of the physical activity), the individual responsible for defining the template (e.g., an identifier of the individual, a sex of the individual, a height or weight of the individual), or a session in which the physical activity is performed for definition purposes (e.g., a date or time of the session, a type of computing device used to generate the video). Accordingly, the metadata may specify that “Jane Doe” defined the template for creating a “squat” on “1 Jan. 2023.” Maintaining this information not only allows the motion monitoring platform to readily identify appropriate templates, but also better understand when additional templates or changes to existing templates are necessary. For example, assume that the template for a physical exercise is defined by a male physiotherapist and that the motion monitoring platform discovers, through automated analysis or user feedback, that the template is underperforming for female users. In such a scenario, the motion monitoring platform may prompt creation of another template for the physical exercise that is defined by a female physiotherapist, so that the physical exercise is associated with multiple templates (e.g., one that can be used for male users and one that can be used for female users).

The motion monitoring platform can also implement a template for the purpose of establishing how well an individual is performing the corresponding physical activity. To implement a template for a physical exercise, the motion monitoring platform can initially obtain a video that is representative of a series of frames, in temporal order, in which an individual-who may be a patient, for example-performs a physical activity. The video may be recorded by the individual as part of a session in which she is prompted to perform the physical activity, potentially among other physical activities. Thereafter, the motion monitoring platform can apply, to the video, a pose estimator so as to produce a series of estimated poses, each of which is representative of a pose of the individual in a corresponding one of the series of frames. For each of the estimated poses, the motion monitoring platform can compare that estimated pose to some or all of the states defined in the template and then identify a given state that is most similar to that estimated pose. By doing this in an ongoing manner, the motion monitoring platform can establish, in real time, a current state of the individual in performing the physical activity. Based on the current state, the motion monitoring platform can identify appropriate feedback to convey to the individual. Because this feedback is tailored to the current state, it is more likely to effective in achieving its goal (e.g., improving performance of the physical activity or improving adherence to a program requiring completion of sessions over time).

### Processing System

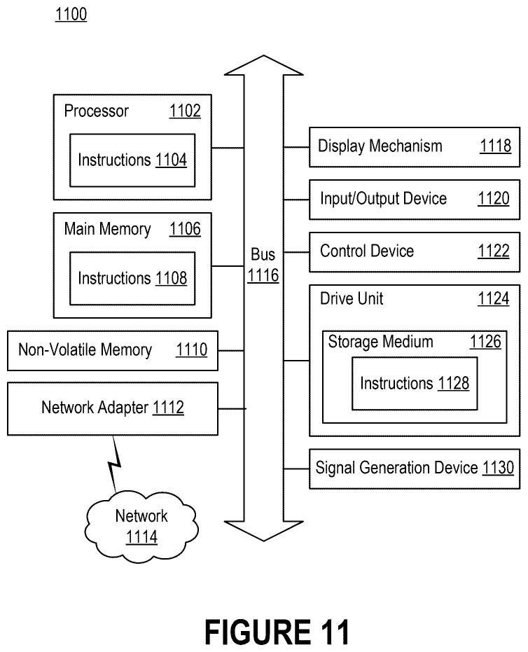

FIG. 11 includes a block diagram illustrating an example of a processing system 1100 in which at least some operations described herein can be implemented. For example, components of the processing system 1100 may be hosted on a computing device that includes a motion monitoring platform (e.g., motion monitoring platform 202 of FIG. 2 or motion monitoring platform 312 of FIG. 3).

The processing system 1100 can include a processor 1102, main memory 1106, non-volatile memory 1110, network adapter 1112, video display 1118, input/output devices 1120, control device 1122 (e.g., a keyboard or pointing device such as a computer mouse or trackpad), drive unit 1124 including a storage medium 1126, and signal generation device 1130 that are communicatively connected to a bus 1116. The bus 1116 is illustrated as an abstraction that represents one or more physical buses or point-to-point connections that are connected by appropriate bridges, adapters, or controllers. The bus 1116, therefore, can include a system bus, a Peripheral Component Interconnect (“PCI”) bus or PCI-Express bus, a HyperTransport (“HT”) bus, an Industry Standard Architecture (“ISA”) bus, a Small Computer System Interface (“SCSI”) bus, a Universal Serial Bus (“USB”) data interface, an Inter-Integrated Circuit (“I2C”) bus, or a high-performance serial bus developed in accordance with Institute of Electrical and Electronics Engineers (“IEEE”) 1394.

While the main memory 1106, non-volatile memory 1110, and storage medium 1126 are shown to be a single medium, the terms “machine-readable medium” and “storage medium” should be taken to include a single medium or multiple media (e.g., a centralized/distributed database and/or associated caches and servers) that store one or more sets of instructions 1128. The terms “machine-readable medium” and “storage medium” shall also be taken to include any medium that is capable of storing, encoding, or carrying a set of instructions for execution by the processing system 1100.

In general, the routines executed to implement the embodiments of the disclosure can be implemented as part of an operating system or a specific application, component, program, object, module, or sequence of instructions (collectively referred to as “computer programs”). The computer programs typically comprise one or more instructions (e.g., instructions 1104, 1108, 1128) set at various times in various memory and storage devices in a computing device. When read and executed by the processor 1102, the instruction(s) cause the processing system 1100 to perform operations to execute elements involving the various aspects of the present disclosure.

Further examples of machine- and computer-readable media include recordable-type media, such as volatile memory devices and non-volatile memory devices 1110, removable disks, hard disk drives, and optical disks (e.g., Compact Disk Read-Only Memory (“CD-ROMs”) and Digital Versatile Disks (“DVDs”)), and transmission-type media, such as digital and analog communication links.

The network adapter 1112 enables the processing system 1100 to mediate data in a network 1114 with an entity that is external to the processing system 1100 through any communication protocol supported by the processing system 1100 and the external entity. The network adapter 1112 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### REMARKS

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments can vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A method performed by a computer program executing on a computing device, the method comprising:
obtaining a digital image that includes an individual who has been prompted to perform a physical activity via an interface;
applying, to the digital image, a machine learning model that is trained to produce, as output, an estimated pose of the individual;
comparing the estimated pose to a template associated with the physical activity, wherein the template includes—
(i) a first reference pose that corresponds to a relaxed position and
(ii) a second reference pose that corresponds to an engaged position;
in response to a determination that the estimated pose is more statistically similar to the second reference pose than the first reference pose,
determining, based on the estimated pose, a current state of the individual with respect to performance of the physical activity;
identifying appropriate feedback for the individual based on the current state; and
causing digital presentation of the appropriate feedback on the interface.

2. The method of claim 1,
wherein the current state is determined from among a set of states, and
wherein each state in the set of states corresponds to a different temporal position and/or a different spatial position with reference to the engaged position.

3. The method of claim 2, wherein the set of states includes—
(i) a first state that is representative of the individual moving into the engaged position,
(ii) a second state that is representative of the individual having achieved a valid pose for the physical activity, and
(iii) a third state that is representative of the individual moving out of the engaged position.

4. The method of claim 2, wherein the set of states includes—
(i) a first state that is representative of the individual moving into the engaged position,
(ii) a second state that is representative of the individual having achieved a valid pose for the physical activity but not having achieved a personal maximal engagement,
(iii) a third state that is representative of the individual having achieved the personal maximal engagement, and
(iv) a fourth state that is representative of the individual moving out of the engaged position.

5. The method of claim 1, wherein said determining is performed by a multi-state machine that is programmed to recognize and classify repetitive movement between the first and second reference poses.

6. The method of claim 1, wherein the digital image is generated by a camera included in the computing device.

7. The method of claim 1,
wherein the estimated pose is representative of a collection of predicted locations for anatomical regions of the individual,
wherein the first reference pose is representative of a first predetermined arrangement of the anatomical regions, and
wherein the second reference pose is representative of a second predetermined arrangement of the anatomical regions.

8. The method of claim 7, wherein statistical similarity between the estimated pose and each of the first and second reference poses is determined by computing, for each of the anatomical regions,
(i) a first score that is indicative of distance between a predicted location in the estimated pose and a corresponding location in the first reference pose, and
(ii) a second score that is indicative of distance between the predicted location in the estimated pose and a corresponding location in the second reference pose.

9. A non-transitory medium with instructions stored thereon that, when executed by a processor of a computing device, cause the computing device to perform operations comprising:
obtaining a video that is representative of a series of frames, in temporal order, in which an individual performs a physical activity;
applying, to the video, a machine learning model so as to produce a series of estimated poses, each of which is representative of an estimate of a pose of the individual in a corresponding one of the series of frames;
deriving a template for the physical activity based on the series of frames, wherein the template includes—
(i) a first reference pose that corresponds to a relaxed position and
(ii) a second reference pose that corresponds to an engaged position; and
storing the template in a data structure.

10. The non-transitory medium of claim 9, further comprising:
associating metadata with the data structure that specifies a characteristic of the physical activity, the individual, or a session in which the physical activity is performed.

11. The non-transitory medium of claim 10, wherein the characteristic is a type of the physical activity, an intensity of the physical activity, an identifier of the individual, a date of the session, or a type of computing device used by the individual to generate the video in the session.

12. A non-transitory medium with instructions stored thereon that, when executed by a processor of a computing device, cause the computing device to perform operations comprising:
obtaining a video that is representative of a series of frames, in temporal order, in which an individual performs a physical activity;
applying, to the video, a machine learning model so as to produce a series of estimated poses, each of which is representative of an estimate of a pose of the individual in a corresponding one of the series of frames;
for each estimated post in the series of estimated poses,
comparing that estimated pose to a template associated with the physical activity, so as to continually establish a current state of the individual with respect to performance of the physical activity; and
presenting feedback to the individual at least one during the performance of the physical activity,
wherein the feedback is generated or selected based on the current state of the individual.

13. The non-transitory medium of claim 12, wherein the template includes multiple states, each of which is associated with a different one of multiple reference poses.

14. The non-transitory medium of claim 12, wherein said comparing results in that estimated pose being compared against each reference pose of the multiple reference poses, so as to produce multiple metrics indicative of similarity.

15. The non-transitory medium of claim 14, wherein the current state is established based on whichever of the multiple reference poses is determined to be most similar to that estimated pose, as determined based on the multiple metrics.
